(19) United States
(12) Patent Application Publication
(10) Pub. No.: US 2018/0192937 A1
(43) Pub. Date: Jul. 12, 2018

(54) APPARATUS AND METHOD FOR DETECTION, QUANTIFICATION AND CLASSIFICATION OF EPIDERMAL LESIONS

(71) Applicant: LINKVERSE S.R.L., Roma (IT)

(72) Inventors: Andrea CHERUBINI, Roma (IT); Nhan NGO DINH, Roma (IT)

(21) Appl. No.: 15/746,854

(22) PCT Filed: Jul. 25, 2016
(86) PCT No.: PCT/IB2016/054414

§ 371 Date: Jan. 23, 2018

(30) Foreign Application Priority Data
Jul. 27, 2015 (IT) 102015000038617

(51) Int. Cl. A61B 5/00; G06T 7/00

(52) U.S. Cl. CPC A61B 5/445; A61B 5/0064; G06T 7/0014; A61B 5/0077; A61B 5/0071; G06T 2207/10024; G06T 2207/30088; A61B 5/004; A61B 5/70; G06T 2207/10016; A61B 5/7253

(57) ABSTRACT

An apparatus for recording skin lesions on a patient comprises an apparatus for acquiring images and a control and processing unit connected to the apparatus so as to acquire and process a plurality of images of the patient positioned in different angular positions with respect to the apparatus. The control and processing unit comprises a processing block which receives at its input the plurality of images acquired by the apparatus and provides at its output a two-dimensional image obtained as a planar development of a three-dimensional image of the patient calculated in the processing block from the plurality of images acquired. By means of extraction of characteristics indicative of the lesions from the two-dimensional image it is possible to classify the lesions in an automatic or semi-automatic manner. A method for electronically recording skin lesions on a patient is also described.

The present invention relates to an apparatus and to a method for detection, quantification and classification of epidermal or skin lesions. In particular the lesions may be of the acne type. Moreover, the skin zone may be advantageously that of the face. However, here the term “lesion” will be understood as meaning also any superficial alteration of the skin, such as moles, freckles or the like.

In the dermatological sector there exists the need for apparatuses which are able to detect in an objective manner skin lesions, in order, for example, to display them objectively and/or perform automatic or semi-automatic classification and/or quantification thereof and optionally carry out a comparison of their evolution over time.

Apparatuses which record a skin zone of interest in order to obtain an image which is processed digitally so as to show characteristics of this zone have been proposed Usually, however, processing does not allow automatic cataloguing or quantification, but it is useful only as an aid for the dermatologist. Moreover, in the case of relatively large skin zones a single image is not sufficient to provide a useful illustration of the lesions present. For example, in the case of acne, the lesions are usually distributed over the whole zone of the face and a single image, for example front or side image, would give only a partial illustration of the state of the patient's skin.

Some known systems provide the possibility of recording a number of images of the patient's face from various predefined angles. At each predefined angle, the camera records an image. The known apparatus provides, therefore, a sequence of images, one for each predetermined angular position. Recording of images from fixed angles allows for example a comparison of the recorded images at a later time so that it is possible to verify for example the effectiveness of a treatment and/or the evolution of the lesions over time.

It is thus possible to compare each image with the image taken previously or afterwards from the same angle. The operation is however complicated by the fact that comparisons must be made for each single image taken from a certain well-defined angular position.

In order to ensure that the recordings are always carried from the same angle for the various parts of the face, in general supports are needed where the patient is able to rest his/her head (for example in the chin or forehead zone), these supports blocking the movement in precise positions which have been predefined for the recording of each image.

The need to use a support for positioning the patient's head in the exact recording positions is, however, not only bothersome for the patient, but may also alter the final result owing to distortion or masking of the skin in the support zones or in the vicinity of the support zones.

When one considers that a single session for recording the state of the skin on the face may require the recording, processing and comparison of as many as ten or so separate images from different angles, for each of which several digital filtering and processing operations may be required, it can be understood how demanding the procedure may be in terms of the processing power required and time taken.

Even the simple direct comparison by the doctor of entire sequences of images recorded at successive moments may be problematic and give rise to errors.

In the case where it is required to perform the cataloguing and/or classification of the lesions in an automatic or semi-automatic manner, any masking, distortion and the need to deal with a large number of images taken from different well-defined directions make the end result both imprecise and very complex from a computational point of view.

WO2013/106794 describes a radiotherapy system for curing skin tumors, where an ultrasound apparatus obtains 3D images of the tumoural mass and processes them in order to allow positioning of the radiotherapy head. Processing of the three-dimensional images is used to obtain 2D images or “slices” of the three-dimensional mass of the tumor, as acquired by the ultrasound apparatus. No solution is provided, however, as regards examination of the surface.

WO2009/145735 describes the acquisition of images of a patient's face from several positions for the diagnosis of skin diseases. Various methods for ensuring the uniformity of the illumination and pixel colors of the recorded images are described. No system is instead described for spatial processing of the images taken.

US2011/211047 describes a system which acquires different images of the face using different lighting conditions in order to obtain therefrom a processed image with useful information regarding the patient's skin. The processed image may also be displayed as a 3D model of the patient's head. Displaying of a 3D image, however, does not help the doctor with cataloguing or comparison of the processed images.

The general object of the present invention is to provide an apparatus and a method for detection, quantification and classification of epidermal lesions which are able to simplify or improve both the manual procedures and the automatic or semi-automatic procedures, for example providing in a rapid and efficient manner the possibility of displaying, comparing or cataloguing the skin lesions of interest.

In view of this object the idea which has occurred according to the invention is to provide a method for electronically detecting skins lesions on a patient based on images of said patient, comprising the steps of acquiring a plurality of images of the patient from different angular positions and processing this plurality of images so as to obtain a two-dimensional image as a planar development of a three-dimensional image of the patient calculated from the plurality of images acquired.

Still according to the invention the idea which has occurred is to provide an apparatus for detecting skin lesions on a patient, comprising an apparatus for acquiring images and a control and processing unit connected to the apparatus for acquiring and processing a plurality of images of the patient in different angular positions with respect to the apparatus, characterized in that the control and processing unit comprises a processing block which receives at its input the plurality of images acquired by the apparatus and provides at its output a two-dimensional image obtained as a planar development of a three-dimensional image of the patient calculated in the processing block from a plurality of acquired images.

In order to illustrate more clearly the innovative principles of the present invention and its advantages compared to the prior art, an example of embodiment applying these principles will be described below with the aid of the accompanying drawings. In the drawings:

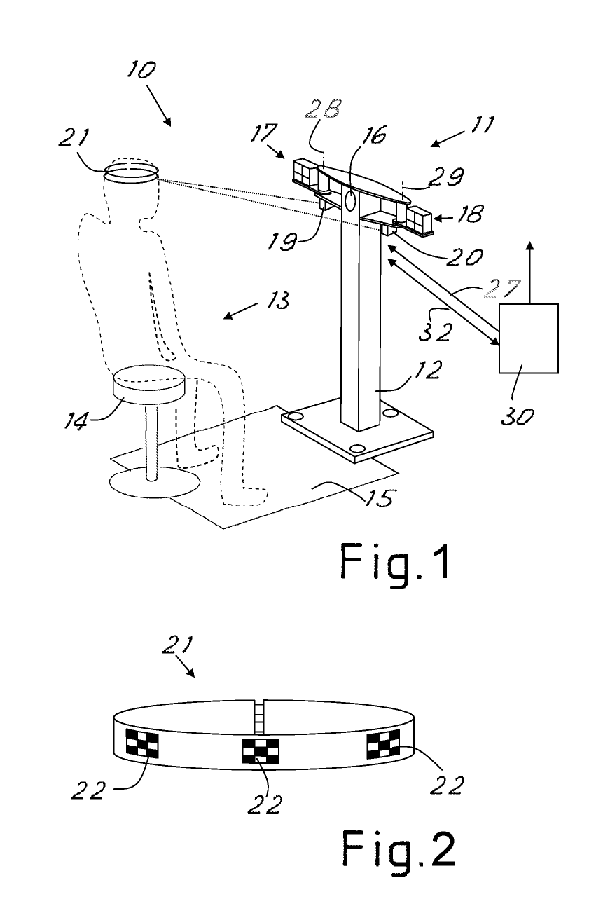

FIG. 1 shows a schematic perspective view of an apparatus provided in accordance with the invention;

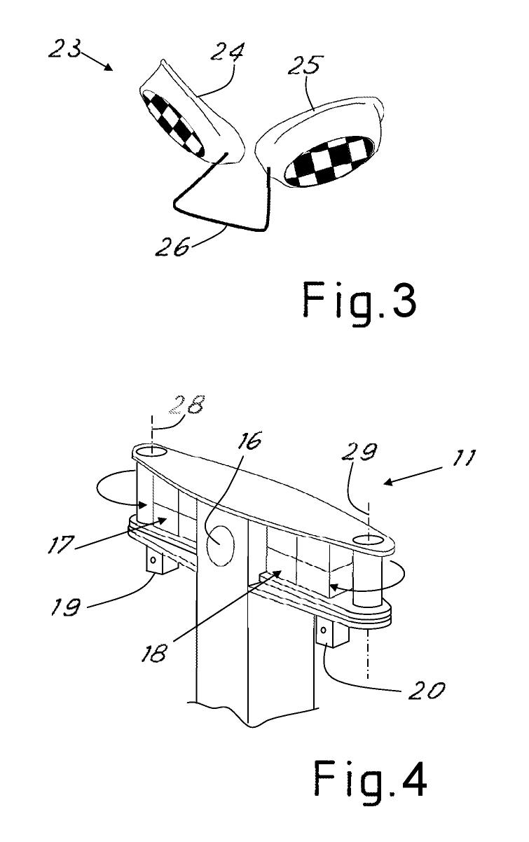

FIGS. 2 and 3 show possible accessories to be worn by the patient during use of the apparatus according to the invention;

FIG. 4 shows a view, on a larger scale, of a part of the apparatus of FIG. 1 in a rest position;

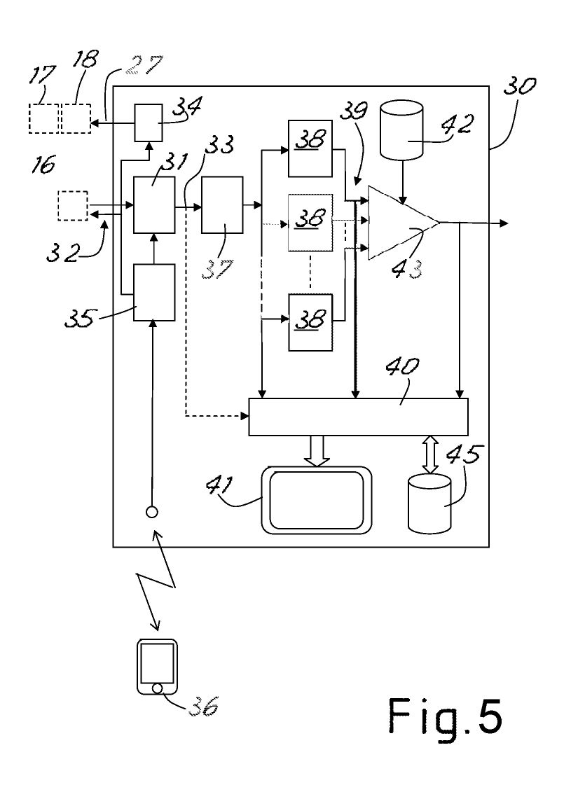

FIG. 5 shows a schematic block diagram of the apparatus according to the invention;

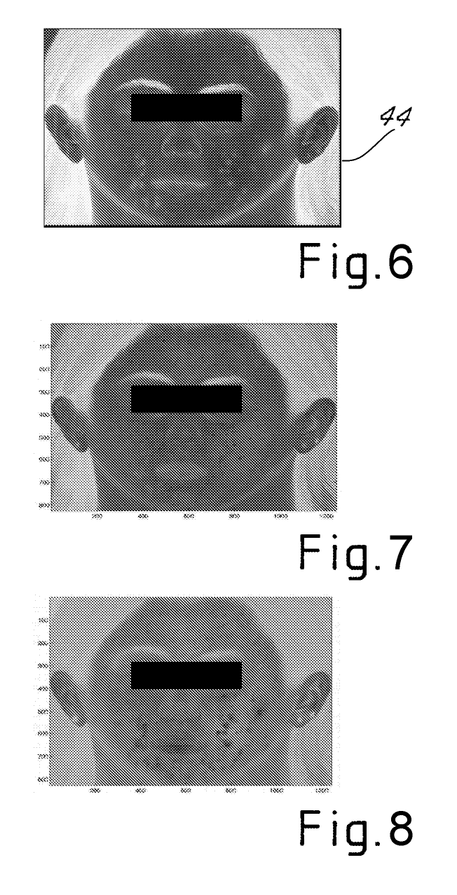

FIGS. 6, 7, 8, 9 and 10 show views of possible images or maps produced by the system according to the invention (the black bands over the eyes are added in the present document to protect the privacy of the patient).

With reference to the figures, FIG. 1 shows an apparatus—denoted generally by 10—which is provided in accordance with the principles of the present invention.

This apparatus 10 comprises an apparatus for acquiring the images 16 (for example, a suitable digital photocamera) advantageously mounted on a recording head 11, preferably supported on the ground by means of a base 12 and arranged opposite a patient station or area 13, preferably provided with a seating element 14 (for example a chair or stool) so that the patient may remain sat in the correct position opposite the recording head 11. The distance between the patient station and the recording head may be preferably predefined (for example 1m). In order to maintain the distance a suitable constraining system may be provided on the ground (for example a footplate 15) arranged between base 12 and seating element 14. The seating element 14 is also advantageously adjustable heightwise so as to adapt the height of the patient to the height of the recording head 11.

In addition to the apparatus 16, the recording head 11 may comprise advantageously illuminators for illuminating the zone to be detected/recorded. These illuminators may consist of a pair of illuminators 17, 18 which are arranged preferably on the two sides of the acquisition apparatus 16 so as to prevent the formation of bothersome shadows on zones of the patient recorded. Each illuminator may comprise one or more light sources. Below, for the sake of simplicity, these light sources will be referred to as being of the “flashlight” type (this representing an advantageous embodiment thereof), even though it is understood that other types of light source may be used (for example a continuous light source).

It has been found to be advantageous if each illuminator comprises at least one light source with a linear filter for polarization of the light and if, in front of the acquisition apparatus 16 there is a suitable linear polarization filter with 90° degree polarization relative to the flashlight filter. For example, the linear polarization filter on the flashlight may have horizontal polarization and the filter on the acquisition apparatus may have vertical polarization.

In this way, by activating the pair of polarized flashlights, it is possible to acquire an image with cross-polarization. As is known, this results in almost complete elimination of the light waves, unless the illuminated body modifies, because of its optical and structural characteristics, the oscillation polarity of the reflected light. This allows essentially characteristics of the illuminated surface to be highlighted, eliminating the surface reflections from the image.

Preferably, each illuminator also comprises a non-polarized flashlight so as to be able to acquire a natural comparison image for the purposes which will be clarified below.

A further flashlight in each illuminator may be advantageously provided with the same polarization as that of the filter on the acquisition apparatus 16. In this way, it is possible to acquire an image with parallel polarization which is useful, for example, for highlighting the brightness of the skin, namely the surface reflections thereon, and which may provide information about a number of is properties, for example the amount of sebum present.

The use of polarized lights thus allows for example the specular surface reflection to be distinguished from the diffused reflection below the skin.

The pairs of flashlights with no polarization, polarization parallel to the filter on the apparatus 16, cross-polarization with the respect to the filter on the apparatus 16 may be activated in sequence so as to obtain the different types of image useful for the subsequent processing operations, as will become clear below.

One or more flashlights in the illuminators may also have an emission band which extends or is comprised within the infrared and/or ultraviolet range, so as to obtain also the acquisition of images at these wavelengths by means of the choice of an acquisition apparatus which is suitably sensitive thereto.

The ultraviolet waveband may be used advantageously in connection with any fluorescence phenomena and thus provide further information about the state of the skin.

For example, in the case of acne, the bacteria present in the lesions are weakly fluorescent in response to ultraviolet light and, as a result, it is possible to obtain ultraviolet images providing further information about the lesions.

In the case of weak fluorescence it has been found to be advantageous to operate the flashlights with ultraviolet emission in fast on/off cycles (for example at a frequency in the region of 10 Hz for a few seconds), acquiring the images during these on/off cycles and carrying out a suitable statistical analysis of the images in order to improve the signal/noise ratio and thus intensify the fluorescent image.

It is advantageous to have flashlights which emit in the waveband of interest and a wide-band recording apparatus, instead of placing an optical filter in front of the lens of the recording apparatus, because the power of the flashlights is such that they reduce substantially the influence of the ambient light and it is not necessary to eliminate entirely or strictly control the ambient light.

It is preferable, however, that the power of the flashlights should be such as to minimize in any case the influence of the ambient light (which may be attenuated).

The flashlights may be for example of the Xenon tube type, preferably with a power of about GN58 and duration of the light pulse in the region of 3 milliseconds at full power. If they must emit also in the infrared range it is possible to use commercial flashlights from which the filters for the visible waveband have been removed. In any case, preferably each type of flashlight in one illuminator is combined with the same type of flashlight in the other illuminator, such that they are made to flash in right-hand/left-hand pairs.

Advantageously, the recording head may also be provided with two luminous pointers 19, 20, for example of the LED type, so as to allow suitable alignment between the recording head and the patient present in the station 13 so that the part of the patient to be examined is situated approximately in the centre of the image acquired.

The apparatus 10 also comprises an electronic control and processing unit 30 which is connected to control the acquisition apparatus 16 and the illuminators 17, 18.

As will become clear below, this unit 30 may be connected to or comprise a user interface which allows the introduction of commands by the operator and displaying of the results of the recording and processing operations. This user interface may be advantageously provided in the form of a personal computer, a suitably programmed tablet or a special dedicated system, or a combination of the two devices.

The unit 30 is advantageously designed to acquire a plurality of images of the patient from different angular positions, so as to allow the processing operations which will be described below.

Preferably, the various angular recording positions are obtained simply by asking the patient to assume suitable different positions in front of the recording head and acquiring a fixed image in each position. This may be obtained by means of a guided procedure which consists in asking the patient to move so as to assume, freely, various more or less predefined positions and acquiring one or more fixed images in each of these positions.

For example, for an examination of the patient's face, the positions may be advantageously a first set of 9 positions simply obtained by asking the patient to rotate his/her head so as to look up and to the right, upwards, up and to the left, to the left, towards the center, to the right, down and to the right, downwards and down and to the left.

For greater certainty the patient may also be asked to assume a second set of 5 positions, by way of confirmation, corresponding to only the directions: upwards, left, centre, right and downwards.

In any case, it has been found that even only one set of five positions, or even only one set of three positions, may be sufficient for correct processing, owing to the principles of the present invention.

For each position it is possible to acquire, preferably in sequence, images each taken with a different pair of flashlights.

For example, with the three types of flashlights mentioned above it is possible to obtain three sets of 9+5=14 images. The multiple images in each position may be acquired in a sufficiently short time interval (for example within a second) so as not to overly stress the patient or ask him/her to move. It is thus possible to obtain for each position a set of fixed images taken within 1 second, namely for example:
a cross-polarized image taken with the first pair of flashlights
a parallel polarized image taken with the second pair of flashlights
a non polarized image taken with the third pair of flashlights.

As is obvious from the description provided here, for correct positioning of the patient it is also possible to use a virtual positioning mechanism. The user interface shows the real-time video of the patient who, for example, by wearing a marker of known size and shape (such as a headband with a rectangular target symbol arranged on it), gives the operator the possibility of suitably adjusting the distance and the orientation of the patient's face so that the aforementioned target symbol is perfectly aligned within the markers shown superimposed.

The images taken in the various positions and belonging to a same type (namely taken with the same pair of flashlights) may be used to reconstruct a 3D image of the patient's face.

In fact, as is known to the person skilled in the art, in the case where several images of a same object taken from different directions are available, it is possible to employ computational stereopsis, i.e. that series of known algorithms which allow one to obtain information regarding the depth (i.e. the three-dimensional structure) of the object by using two-dimensional images which show the object from different directions.

So-called “stitching” is one of the known reconstruction algorithms which, based on two-dimensional images showing the object from different angles in order to obtain information about the depth (and therefore the three-dimensional structure) of the object, can be used to reconstruct a single two-dimensional image which takes into account the recording angles and other parameters used in the single images.

Stitching includes all those known techniques which, by pinpointing characteristics, attempt to align the various images taken so as to reduce the differences in pixel superimposition. In these techniques, editing of the images involves remapping of the images so as to obtain from them a single panoramic image as end result. Also the differences in color are recalibrated between the single images in order to compensate for the differences in exposure (color mapping). The blending procedures are therefore carried out so as to reduce the unnatural effects and the images are joined together along stitching lines which are optimized to maximize the visibility of the desirable characteristics of the resultant image.

Although stitching or stereopsis may be used even only with two different images, by using several images it is possible both to reduce the background noise and increase the useful signal for the subsequent processing operations and to reduce or eliminate entirely zones in the three-dimensional image obtained which are obscured or are not visible.

With these techniques precise positioning of the patient during the various image recording sequences is not necessary.

In order to obtain in any case, with less computational difficulty, a precise and detailed three-dimensional reconstruction from the plurality of recorded images, it has been found to be advantageous to use suitable markers positioned on the patient to be recorded. As may be now easily imagined by the person skilled in the art, based on the apparent distortion of the markers in the various images of different positions, it is in fact possible to apply the appropriate corrective algorithms, known per se, for three-dimensional reconstitution.

The markers (for example formed by black-and-white checkered patterns) may be advantageously placed on a headband 21 worn by the patient, making sure that zones of interest for the analysis (for example the forehead) are not covered over.

FIG. 2 shows for example in schematic form a possible embodiment of such a headband 21 with markers 22 placed on its external circumference. The headband may be slightly elastic so as to remain firmly in position on the head.

The markers may also be advantageously arranged not directly on the headband, but on suitable projections mounted on the headband (preferably projecting above the head). These markers may be arranged on either side so that at least one of the two markers is visible when the patient's head is turned.

In addition or alternatively, markers may also be placed on a pair of protection pieces 23 for the patient's eyes, as shown in schematic form in FIG. 3. These protection pieces may be useful for preventing the patient from being dazzled by the flashlights and as a protection in the event of ultraviolet light being emitted.

The protection pieces 23 may for example consist of two protection cups 24, 25 (one for each eye) connected by means of an elastic bridge-piece 26.

Especially in the case where other parts of the body are examined, suitable self-adhesive markers may be used for example.

The markers may also be details which are already normally present in the image taken and which may be identified by the system as reference points. For example, markers on the image may be formed by characteristics present in all faces, such as corners of the mouth, ends of the eyes, tip of the nose, eyebrows, etc.

In an advantageous manner, facial recognition algorithms, well-known to the person skilled in the art, may be used for recognition of these markers; alternatively, a supervised learning procedure may be used where the markers are manually drawn by an expert on a limited number of images and are then used to train the expert algorithm so that they may be used later on new images.

The two-dimensional image resulting from the reconstructions or the planar development may be conveniently adapted by means of a model or “template” (which as described below may be provided in the form of a suitable transformation matrix), so as to obtain always substantially identical spatial dimensions and/or resolution, for example so that the markers or key points in this image have predefined Cartesian coordinates.

In this way, the faces of different persons, or of the same person, recorded at different times will be spatially transformed by the prechosen method, but will produce results which are always correlatable, with images where the identical part of the face (for example the right corner of the mouth) will always be positioned at the same coordinates in the image obtained from the planar development.

In other words, the planar development of the 3D image will take into account the position of key points defined by the type of initial image (for example face) in a generic model or predetermined template. In the image obtained from the planar development, these key points will be made to coincide with the position of the corresponding key points in the template. In this way the planar development will be “standardized”, thus making it very easy to compare an identical part of the skin of different persons or of the same person at different times, since it will be sufficient to compare the signal (or part of the image) derived from the images with identical Cartesian coordinates, as will become clear below and from the accompanying figures (FIGS. 6-10).

As can be seen more clearly in FIG. 4, the recording head may be formed by arms supporting the illuminators which project on opposite sides of the acquisition apparatus 16 and which can be advantageously folded towards each other (for example about respective vertical axes 28 and 29) so as to reduce the overall dimensions of the head when not in use.

Alternatively, the illuminators may also be arranged on separate independent mounts arranged on the sides of the support of the acquisition apparatus.

FIG. 5 shows in schematic form the structure of an advantageous embodiment of the control and processing unit 30.

This unit 30 comprises a three-dimensional reconstruction block 3 which receives at its input 32 the images recorded by the acquisition apparatus 16. This block 31 will store the images and carry out a computational stereopsis or stitching using techniques known per se so as to emit at the output 33 the data of a three-dimensional representation obtained from the composition and processing of the sum of the single images. If sequences of images in different conditions are recorded, the block 31 may carry out a three-dimensional processing of each condition (for example a three-dimensional representation in infrared light, visible light, with or without reflections, ultraviolet light, etc.), thus providing the 3D data for each desired recording condition.

The various flashlights of the illuminators are in turn controlled by the output 27 of an illumination control block 34. In order to obtain the various sequences of images to be combined in the three-dimensional processing operations, the three-dimensional reconstruction block 31, the acquisition apparatus and the illumination control block 34 are in turn connected to a management block 35 which performs the flash and recording sequences at predetermined times and based on predetermined parameters. The management block 35 is advantageously connected to a control unit 36 which allows the operator to signal to the management block 35 when the patient is positioned correctly for acquisition of one image of the series of images to be acquired.

Advantageously, the control unit 36 may be a tablet which is suitably programmed and may be connected to the management block 35 by means of a wireless (for example Bluetooth or Wi-Fi) connection.

The 3D data produced at the output 33 of the three-dimensional reconstruction block 31 is sent to a spatial transformation block or smoothing block 37. This block 37 applies a further spatial transformation to the three-dimensional reconstruction obtained from the block 31 based on the sequences of images, so as to map the 3D reconstruction onto a two-dimensional plane, by means of a “flattening” procedure, producing a development in a two-dimensional plane of the three-dimensional reconstruction and carrying out any adaptation of the image as described above by means of a template stored in the block 37, for example as a transformation matrix. The processing block may therefore comprise the predetermined template which is applied so that the two-dimensional image output has the predetermined key points which coincide with the positions of corresponding key points of the template.

Essentially, a suitable transformation matrix is applied to each point x, y, z of the three-dimensional reconstruction of the part of the patient to be examined (in particular the face) in order to map it (identifying in it any suitable key points) in a plane X, Y, using a procedure known per se and able to be easily imagined by the person skilled in the art. In this way it is possible to obtain, for each 3D reconstruction, a single flat image which shows, extended in a plane, the entire surface of the skin which is to be examined. In other words, a “flattened” image is obtained where basically each point of the patient's skin recorded is shown as though it were viewed from a direction perpendicular to the tangent of the surface of the 3D image at that point. This provides a clear, complete and perfectly reproducible view of all the skin lesions present.

A possible result of such a spatial flattening transformation carried out on a face is shown by way of example in FIG. 6 with an image which is indicated generally by 44. In this figure, the processing of the single images has been carried out using the stitching technique. The face is obviously stretched and distorted with respect to the original 3D image and the series of images taken from the various angles, but it contains all the information regarding the skin lesions.

In this figure it is possible to see clearly the application of a template which shows the image stretched to a standard form. It can be seen, for example, that the nose is not distorted as would have been the case if linear “stretching” of the three-dimensional image had been carried out.

In fact, more than one reference template may be applied so as to be able to represent in the best possible manner the three-dimensional development of various zones of the patient's skin. For example, in the case of the face, a template for the entire face, except for the nose (as can be seen in FIG. 6), and a template intended specifically for the nose (as may be now easily imagined) may be used, since the nose in general projects towards the recording line and therefore may require a three-dimensional development and a subsequent development in a dedicated plane, in order to represent it in the best manner possible, without excessive distortion of the adjacent zones.

In the case where illuminators with three pairs of polarized flashlights, and not as described above, are used, the spatial transformation block 37 advantageously analyzes the three series of images input and calculates three flattened maps from the three sets of cross-polarized, parallel polarized and non-polarized images.

The calculation is carried out using known spatial transformation algorithms which may also be advantageously based on the distortion of the checkered patterns of the markers or also on automated recognition of parts of the image (landmarks) and subsequent stitching of the different images, as already described.

The flattened images or “maps” may be sent from the block 37 to a plurality of filtering blocks 38. These filtering blocks perform digital filtering of the images so as to extract from them specific information 39 selected to highlight and/or classify particular skin lesions.

The filtering concept is understood here in the widest sense and the corresponding operation may comprise the application of a wide range of transfer functions. For example, as specified below, filtering may also be performed as a given transformation of the color space of the flattened images output by the block 37. Moreover, the filtering may be performed so as to produce an extraction of geometric parameters, such as the area of the lesions and/or their eccentricity.

For example, FIG. 7 shows the result of a filtering operation which envisages the transformation of the color space of the image shown in FIG. 6 into the color space associated with melanin such a transformation is per se known to the person skilled in the art. The image or map thus obtained therefore contains the information relating to the melanin present in the various points of the face shown in FIG. 6.

Again by way of example, FIG. 8 shows the result of a filtering operation which envisages the transformation of the color space of the image shown in FIG. 6 into the color space associated with hemoglobin. This transformation is also known per se to the person skilled in the art. The image or map thus obtained therefore contains the information relating to the hemoglobin present in the various points of the face shown in FIG. 6. It is obviously possible to map the original images in a different color space which satisfies the needs of the user or which best highlights the contrast characteristics desired in the picture.

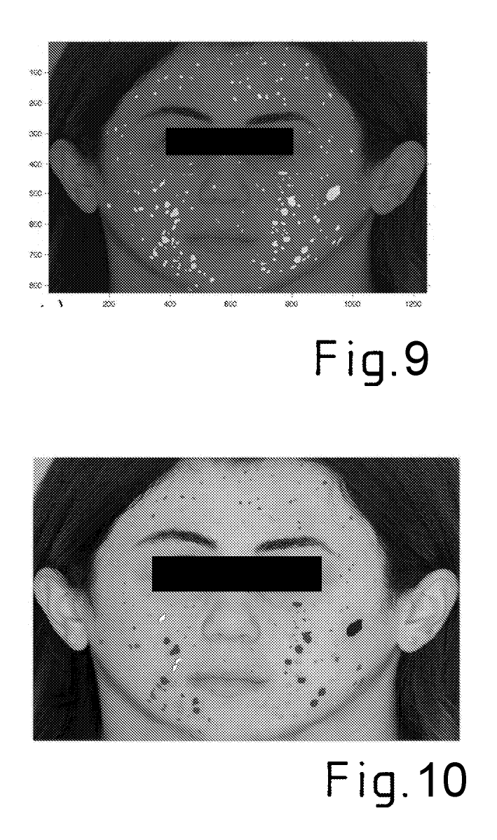

Again by way of example, FIG. 9 shows the result of a filtering operation which envisages the extraction of only the information relating to the area of the lesions present on the face shown in FIG. 6. This extraction is also known per se to the person skilled in the art. This extraction may be based, for example on the color variations in the image of FIG. 6, optionally combined with the melanin and/or hemoglobin information resulting from the corresponding filtering operations, so as to define the edges of the lesions. An image or map which shows the areas of interest is thus obtained.

For each area defined it is also possible for example to extract further geometric information, such as that relating to the average diameter, the eccentricity of the area (namely, for example the relative proportion of the smaller and larger orthogonal axes of the area), etc.

In a convenient manner, as a result of the flattened image of the present invention, the quantitative data may be easily related to single lesions, identified manually or automatically, or to a series of predefined regions which correspond always to the same area in different patients. The size and shape of these areas may be defined as required, for example, but not solely, by means of horizontal and vertical lines which intersect the flattened face at regular distances, creating a grid. From a comparison of quantitative values extracted from the elements of the image contained in a subarea of the grid it is easy to evaluate the temporal progression of a same patient's condition during different visits made over time or to make a comparison between different patients.

All the various representations or maps of the patient's face, or a selected subset thereof, may for example be provided to a display interface 40 which displays them on a suitable display 40.

For example, following a command entered by the operator, the interface 40 may display the three-dimensional image (or the three-dimensional images obtained with the various illumination conditions) calculated by the block 31, optionally providing the possibility of rotating the image so as to view it from various view points, or else the flattened image output by the block 37, or the images output by the various filters 39. This allows, for example, an expert (for example a dermatologist) to have multiple useful information about the state of the patient's skin by locating specific characteristics shown in the various processed images. The further possibility of memorizing for each patient the images obtained, by storing them in a suitable electronic memory 45, also allows a visual comparison to be made between images obtained at successive points in time for the same patient, so as to obtain for example information about the evolution of a pathology or allow objective quantification of the efficacy or otherwise of a treatment.

Owing to the use of single “flattened” images as described above, it is possible to make an easy and rapid direct comparison between two simple images obtained at different times and reproduced with the same identical orientation, instead of comparing a plurality of images taken from different angles using the known methods.

The characteristic parameters obtained by means of the various filtering operations carried out on the initial image (or initial images) at various points on the image may be used to classify the lesions of interest. These parameters constitute essentially a “fingerprint” or “signature” for the various classes of lesions to be defined.

For example, in the case of acne lesions, the pustules have an intensity peak in the white region and a high standard deviation in the hemoglobin histogram, while the papules have a high degree of homogeneity in the melanin histogram (namely a low standard deviation) and very different hemoglobin and melanin values. Using these parameters as characteristic parameters it is therefore easy to distinguish between two types of lesion.

Generally, the greater the number of classes which are to be distinguished, the greater will be the number of characteristic parameters useful for assigning with sufficient precision the lesions to the respective classes.

In the case of lesions due to acne vulgaris, for example the lesions may be subdivided into five classes, namely: open blackheads, closed blackheads, papules, pustules and cysts. If desired, moles or skin blemishes may also be recorded.

The characteristic parameters may advantageously be or comprise at least: the area, the diameter, the eccentricity, the melanin fraction, the hemoglobin fraction. The diameter and the area may, for example, be expressed in pixels, after a suitable calibration of the recordings.

As will be further clarified below, definition values of the various classes may be traced by means of suitable statistical investigations and the initial collaboration of a human expert.

The characteristic parameters chosen in order to define the classes of various types of lesions which are of interest may be stored beforehand in an electronic database 42 present in the system and during the analysis a comparison block 43 may perform the comparison between the indicative parameters associated with each lesion identified in the initial image, and the contents of the database 42, so as to classify automatically the lesions. The search in the database in order to obtain the classification may be easily implemented using known machine learning algorithms.

The definition and classification of the groups in the maps which represent the images enables for example a count and automatic classification of all the skin lesions of interest to be carried out. As mentioned above, the parameters selected for the classification may be multiple, depending on the specific requirements.

In order to enter the parameters into the database an initial machine learning procedure may also be performed. According to this procedure, the images collected from a sufficiently wide sample range of patients may be analyzed so that the system records the predetermined parameters representing each lesion identified. An expert then associates manually the correct class with each lesion defined. In this way the database is initially populated by associating the relevant correct class with a range of values of the parameters.

The system may also have a further self-learning function whereby, during normal use, the expert may enter the correct class for those lesions where the class was not automatically identified or an incorrect class was identified. This increases the statistical basis of the database, such that the system becomes increasingly more efficient with use. The system behaves essentially as an expert system.

During use of the system, the result of classification of the lesions recorded for a patient may be shown in various ways, for example depending on specific requirements.

For example, after scanning the patient, simply the number of lesions identified for each class may be provided. This may, for example, give the doctor an indication of the evolution of the lesions and/or the efficacy of a treatment, or may be useful for documentation or statistical purposes in clinical studies or the like.

In addition or alternatively, it is possible to provide an image in which the lesions identified are highlighted using false colors or in different shades of grey depending on the class to which they belong. Such a representation is shown by way of example in FIG. 10. As will be clear from the description provided above, it is also possible to subdivide the image into a series of predefined areas, and quantitative values may be indicated for each area.

This allows one to obtain for example a graphical representation which can be rapidly consulted and easily compared at a glance with a prior state of the patient which is shown alongside and with a similar graphic representation.

At this point it is clear how the predefined objects have been achieved by providing an apparatus and a method which allow the definition, the cataloguing and the easy and rapid—manual, semi-automatic or automatic—comparison of skin lesions, such as, for example and in particular, those caused by acne.

Obviously the description above of an embodiment applying the innovative principles of the present invention is provided by way of example of these innovative principles and must therefore not be regarded as limiting the scope of the rights claimed herein.

For example, as is clear for the person skilled in the art, the various aforementioned blocks of the electronic control and processing unit may be realized in the form of hardware, software or a combination of hardware and software. In particular, the system may comprise a personal computer or a server which receives the images in digital format by means of a suitable connection to a digital recording apparatus and is programmed to perform via software all the processing functions requested. In the practical embodiment some of the functional blocks described for the unit 30 may also be dispensed with or be replaced or supplemented by other functional blocks.

The functions of the various blocks described above may also be incorporated in a single block or on the contrary further divided up. For example, the series transformation of images acquired, 3D reconstruction and flattened image may be realized in a single mathematical transformation step from acquired images to flattened image, if the 3D image is not required or is of no interest.

The three-dimensional reconstruction block 31 and the flattening block 37 may also be considered as being contained in a processing block 31, 37 which receives the images taken from various angles and provides at its output the “flattened” two-dimensional image. The intermediate product, namely the data 33 of a three-dimensional image, may be supplied or not externally depending on the specific requirements.

Moreover, some or all the blocks may be realized with a distributed system. For example, the first acquisition part may be local to the acquisition system, while the final processing and/or classification may be realized by remote units via a connection network.

In particular, the database 42 containing the “fingerprints” or “signatures” of the lesions may be centralized or be remote so as to contain the statistical results of a large quantity of classifications carried out also by several systems.

As a result the classification becomes more precise and reliable and develops over time as the database increases.

Moreover, the remote system may be used to receive the data obtained from the recordings for a plurality of patients such that, for example, extensive studies may be carried out as to the efficacy of one or more pharmacological treatments. The data may be rendered automatically anonymous before being sent from the acquisition site and this may be advantageous for example in the case of clinical studies.

The local apparatus which comprises necessarily the recording head may also comprise (for example inside the head itself) an access point to which the control tablet 36 connects automatically. It may also be envisaged that the local part of the system collects the biomedical data and the images and sends it to the network in a preferably encrypted and compressed form so as to be received by remote stations for the subsequent processing and storage operations. A local control console may also be provided for receiving notifications, approving the sending of data, displaying the intermediate results of the processing operations or the final result, etc. This control console may be realized for example with an application installed again on the tablet. The illuminators, if considered to be unnecessary, may also be dispensed with or, on the contrary, may be formed by a greater number of light sources, as described above.

Owing to the fact that the images acquired may be used by the system 30 in order to reconstruct a representation with three-dimensional information of the patient, it is also possible to produce two-dimensional artificial images of the patient taken from directions different from the directions in which the plurality of real initial images were recorded. It is thus also possible to define for example standard directions for a “virtual” recording and virtual two-dimensional images may be produced, these appearing to have been recorded from these standard directions. This allows a precise comparison between images of different patients or the same patient recorded at successive moments, without the patient being obliged to assume these precise standard positions in reality. With this system it is also possible to obtain “artificial” images taken from directions which in reality do not exist in the plurality of real images recorded.

As already mentioned, the artificial images may also comprise the image of the planar development of the 3D image obtained by applying a conversion template (or matrix), namely using a template which defines predefined positions for various parts of the reconstructed image.

Moreover, whether a manual comparison or an automatic comparison is to be performed, with the systems of the prior art in general it is not easy to compare the dermatological situation of faces of several persons or also of the same person at a later time, owing to the differences in the form of the face recorded, said differences being due for example both to the different recording angle of the various images and the different person involved.

Owing to application of the principles of the invention, such as the application of a template for the positioning of the various parts of the reconstructed face, it is instead possible according to the invention to obtain an arrangement of the parts of the image related to an “average face”, namely (since the present invention allows the creation of a one-to-one relationship between the 3D face and a 2D map) it is possible to select as a 2D map in a convenient manner a flat version of an average face, in which the main anatomical parts (for example eyes, nose, mouth, ears) are situated along the same x,y coordinates also in the case of different persons. The multiple 2D images obtained from the single images are thus related to this average face so that each portion of the face reconstructed as a planar development of the 3D image is based principally on the recorded image which has the perpendicular situated closest to the ideal perpendicular.

Even though the original spatial proportions of the skin are thus modified compared to the original image recorded, since the transformation is one-to-one it is always possible to retrace the real dimension of the original photograph. Moreover it should not be forgotten that, even in the original images, the sole zones which minimize the parallax errors, are those where the angle of incidence relative to the perpendicular of the face does not exceed a limit value.

All this can be seen also from the comparison of FIGS. 6 to 10 where it can be noted how the main parts of the face are always identically positioned in the image of the planar development of the 3D reconstruction of the various figures, which could also relate to different patients, or to the same patient at different moments in time. As already mentioned above, although for the sake of simplicity reference has been made mainly to a face, the principles of the invention may also be applied to other parts of the body, as may be now easily imagined by the person skilled in the art on the basis of the description provided above.

1. Method for electronically detecting skins lesions on a patient based on images of said patient, comprising the steps of acquiring a plurality of images of the patient from different angular positions and processing this plurality of images so as to obtain a two-dimensional image as a planar development of a three-dimensional image of the patient calculated from the plurality of images acquired.

2. Method according to claim 1, wherein the two-dimensional image is subjected to filtering so as to extract from it given characteristics indicative of the lesions present in the image.

3. Method according to claim 2, wherein the lesions are automatically classified on the basis of the said indicative characteristics extracted.

4. Method according to claim 2, wherein the said indicative characteristics comprise one or more of the following characteristics of a lesion: area, average diameter, eccentricity, melanin fraction, hemoglobin fraction.

5. Method according to claim 2, wherein the classification is performed automatically by means of a search algorithm in an electronic database containing the associations between predefined classes and indicative characteristics.

6. Method according to claim 5, wherein the electronic database is initially populated by means of a machine learning procedure.

7. Method according to claim 1, wherein three-dimensional information about the patient is processed from the plurality of images and from said information an artificial two-dimensional image of the patient is obtained, said two-dimensional image being taken from a definite direction different from the directions in which the plurality of acquired images were taken.

8. Method according to claim 1, wherein the lesions are acne lesions.

9. Method according to claim 1, wherein, in the planar development of the three-dimensional image, predetermined key points of the image are made to coincide with positions of corresponding key points on a predetermined template.

10. Apparatus for detecting skin lesions on a patient, comprising an apparatus for acquiring images and a control and processing unit connected to the apparatus for acquiring and processing a plurality of images of the patient positioned in different angular positions with respect to the apparatus, wherein the control and processing unit comprises a processing block which receives at its input the plurality of images acquired by the apparatus and provides at its output a two-dimensional image obtained as a planar development of a three-dimensional image of the patient calculated in the processing block from the plurality of acquired images.

11. Apparatus according to claim 10, wherein the processing block comprises a predetermined template which is applied by the processing block so that, in the two-dimensional image, predetermined key points coincide with positions of corresponding key points in the predetermined template.

12. Apparatus according to claim 11, wherein the control and processing unit comprises filtering blocks which receive the two-dimensional image and extract from it predetermined characteristics of the lesions contained therein.

13. Apparatus according to claim 12, wherein the control and processing unit comprises a classification block which receives the said predetermined characteristics from the filtering blocks and classifies the lesions on the basis of these characteristics.

14. Apparatus according to claim 11, wherein it comprises a display interface connected to a display for displaying, upon command, the images acquired and/or calculated and processed and/or the cataloguing of the lesions performed.

15. Apparatus according to claim 11, wherein it comprises illuminators for illuminating a recording zone of the acquisition apparatus, the illuminators comprising one or more of the following light sources: polarized light source, ultraviolet light source, infrared light source, and in that any polarized light sources comprise a source with cross polarization relative to a polarized filter placed on the acquisition apparatus and/or a source with parallel polarization relative to this polarized filter placed on the acquisition apparatus.

16. Apparatus according to claim 15, wherein the illuminators are two in number and are arranged on the two sides of the acquisition apparatus.

17. Apparatus according to claim 15, wherein the acquisition apparatus and the illuminators are contained in a recording head having centrally the acquisition apparatus and on the two sides the two illuminators on oppositely projecting arms.

18. Apparatus according to claim 14, wherein sources emitting ultraviolet light emit with on/off cycles and the associated images are statistically processed in order to improve the signal/noise ratio and intensify the fluorescent image.

19. Apparatus according to claim 11, wherein the lesions are acne lesions.
